(19) United States
(12) Patent Application Publication
(10) Pub. No.: US 2017/0263157 A1
(43) Pub. Date: Sep. 14, 2017

(54) ANESTHESIA APPARATUS ADAPTED FOR OPERATING IN ONE OF A GAS- DISPENSING MODE AND A GAS-LESS SIMULATION MODE

(71) Applicant: CAE HEALTHCARE CANADA INC., Saint-Laurent (CA)

(72) Inventor: Hugo AZEVEDO, Saint-Laurent (CA)

(73) Assignee: CAE HEALTHCARE CANADA INC., Saint-Laurent (CA)

(21) Appl. No.: 15/064,345

(22) Filed: Mar. 8, 2016

(30) Foreign Application Priority Data
Mar. 8, 2016 (CA) 2923189

(51) Int. Cl. G09B 23/28; A61M 16/12; A61M 16/10; A61M 16/01

(52) U.S. Cl. CPC G09B 23/28; A61M 16/01; A61M 16/12; A61M 16/104; A61M 2205/505; A61M 2205/27; A61M 2205/3584; A61M 2205/3553; A61M 2205/60; A61M 2230/00

(57) ABSTRACT

Anesthesia apparatus adapted for operating in one of a gas-dispensing mode and a gas-less simulation mode, and method of operating the anesthesia apparatus. A mode selection command is received from an input/output unit of the anesthesia apparatus. A control unit of the anesthesia apparatus selects one of a gas-dispensing mode and a gas-less simulation mode based on the received mode selection command. At least one gas control parameter is received from the input/output unit. When the gas-dispensing mode is selected, the control unit actuates gas dispensing means of the anesthesia apparatus based on the received at least one gas control parameter. When the gas-less simulation mode is selected, the control unit prevents actuation of the gas dispensing means, and transmits the at least one gas control parameter to a patient simulation system via a communication interface of the input/output unit.

### TECHNICAL FIELD

The present disclosure relates to the field of medical education and simulation in healthcare. More specifically, the present disclosure relates to an anesthesia apparatus adapted for operating in one of a gas-dispensing mode and a gas-less simulation mode.

### BACKGROUND

Simulators are used to practice complex and potentially dangerous tasks in a realistic and secure environment. A particular class of simulators comprises medical simulators designed for reproducing human body functions, and are used in the context of medical training.

Anesthesia training is a particular form of medical training, which typically uses a patient simulation system and a real anesthesia machine. The use of a real anesthesia machine and actual anesthetic gases represents a security risk during training if inadvertently used. Furthermore, there are also economic and logistics factors associated with the use of real anesthetic agents in the context of a training involving a real anesthesia machine. For example, in the context of a training session occurring in a classroom, the classroom may not have the appropriate logistics to deal with real anesthetic agents used during the training session.

One way of securely performing anesthesia training is to use in combination a real anesthesia machine and a patient simulation system capable of simulating physiological effects on a patient of a release of anesthetic gases, with the real anesthesia machine being prevented from releasing anesthetic gases during the simulation.

There is therefore a need for an anesthesia apparatus adapted for operating in one of a gas-dispensing mode and a gas-less simulation mode.

### SUMMARY

According to a first aspect, the present disclosure provides an anesthesia apparatus for operating in one of a gas-dispensing mode and a gas-less simulation mode. The anesthesia apparatus comprises gas dispensing means. The anesthesia apparatus also comprises an input/output unit comprising a communication interface, for receiving a mode selection command and receiving at least one gas control parameter. The anesthesia apparatus further comprises a control unit for selecting one of a gas-dispensing mode and a gas-less simulation mode based on the received mode selection command. When the gas-dispensing mode is selected, the control unit further actuates the gas dispensing means based on the received at least one gas control parameter. When the gas-less simulation mode is selected, the control unit further prevents actuation of the gas dispensing means, and transmits the at least one gas control parameter to a patient simulation system via the communication interface.

According to a second aspect, the present disclosure provides a method for operating an anesthesia apparatus in one of a gas-dispensing mode and a gas-less simulation mode. The method comprises receiving a mode selection command from an input/output unit of the anesthesia apparatus. The method comprises selecting, by a control unit of the anesthesia apparatus, one of a gas-dispensing mode and a gas-less simulation mode based on the received mode selection command. The method comprises receiving at least one gas control parameter from the input/output unit. When the gas-dispensing mode is selected, the method further comprises actuating, by the control unit, gas dispensing means of the anesthesia apparatus based on the received at least one gas control parameter. When the gas-less simulation mode is selected, the method further comprises preventing, by the control unit, actuation of the gas dispensing means; and transmitting, by the control unit, the at least one gas control parameter to a patient simulation system via a communication interface of the input/output unit.

According to a third aspect, the present disclosure provides a non-transitory computer program product comprising instructions deliverable via an electronically-readable media, such as storage media and communication links. The instructions comprised in the computer program product, when executed by a control unit of an aesthesia apparatus, provide for operating the anesthesia apparatus in one of a gas-dispensing mode and a gas-less simulation mode, according to the aforementioned method.

### BRIEF DESCRIPTION OF THE DRAWINGS

Embodiments of the disclosure will be described by way of example only with reference to the accompanying drawings, in which:

FIG. 1 illustrates an anesthesia apparatus functioning in a gas-dispensing mode;

FIG. 2 illustrates the anesthesia apparatus of FIG. 1 functioning in a gas-less simulation mode through interactions with a patient simulation system;

FIG. 3 illustrates details of the patient simulation system represented in FIG. 2;

FIG. 4 illustrates a patient simulation system interacting with a plurality of anesthesia apparatus; and

FIGS. 5A and 5B illustrate methods for operating the anesthesia apparatus and the patient simulation system of FIGS. 1, 2 and 3.

### DETAILED DESCRIPTION

The foregoing and other features will become more apparent upon reading of the following non-restrictive description of illustrative embodiments thereof, given by way of example only with reference to the accompanying drawings. Like numerals represent like features on the various drawings.

Various aspects of the present disclosure generally address one or more of the problems related to designing an anesthesia apparatus capable of operating in two modes: a first gas-dispensing mode for dispensing anesthetic gas(es) to a patient (the regular intended use of the equipment), and a second gas-less simulation mode for interaction with a patient simulation system in a medical education (or product demonstration) usability context.

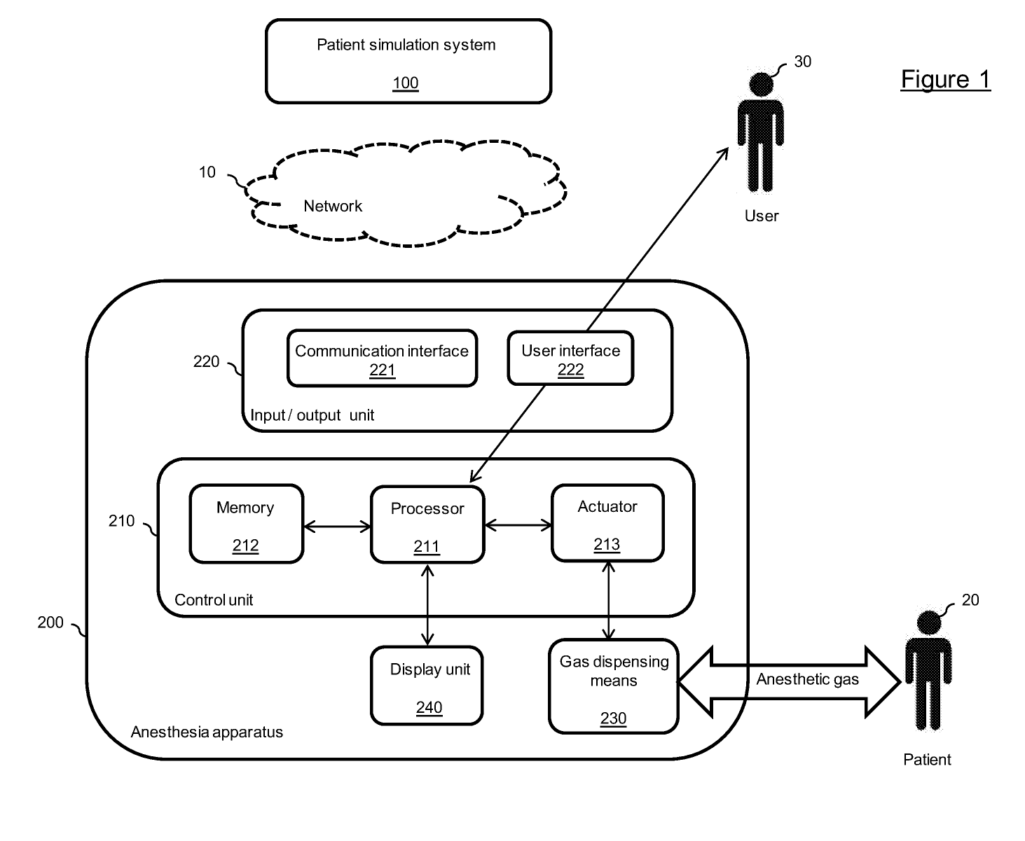

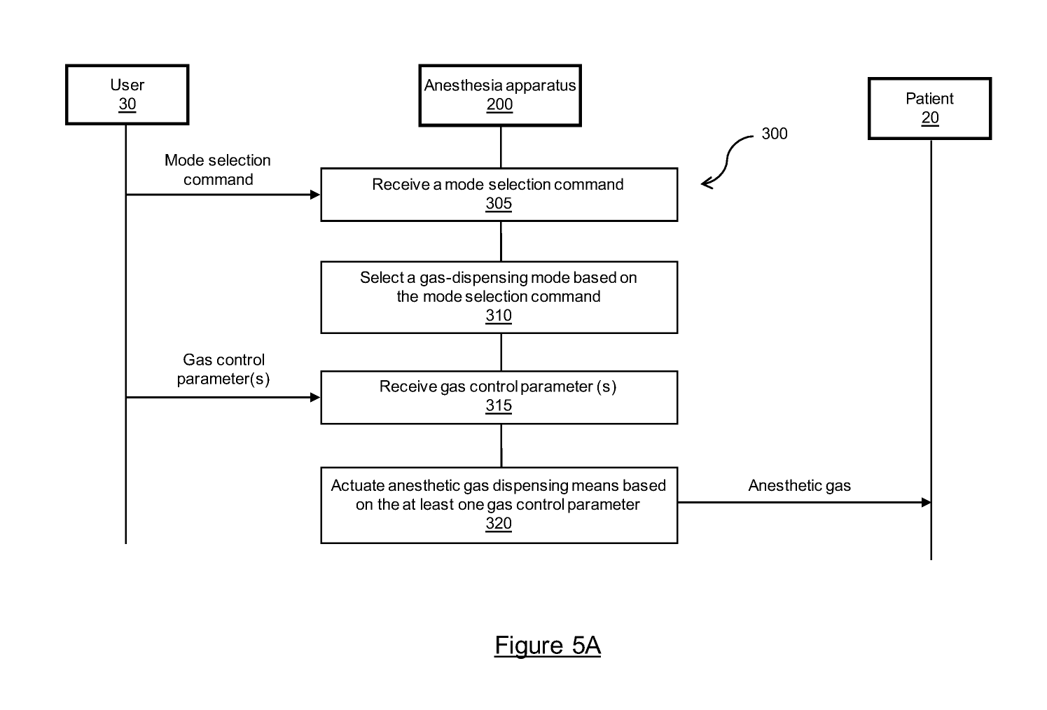

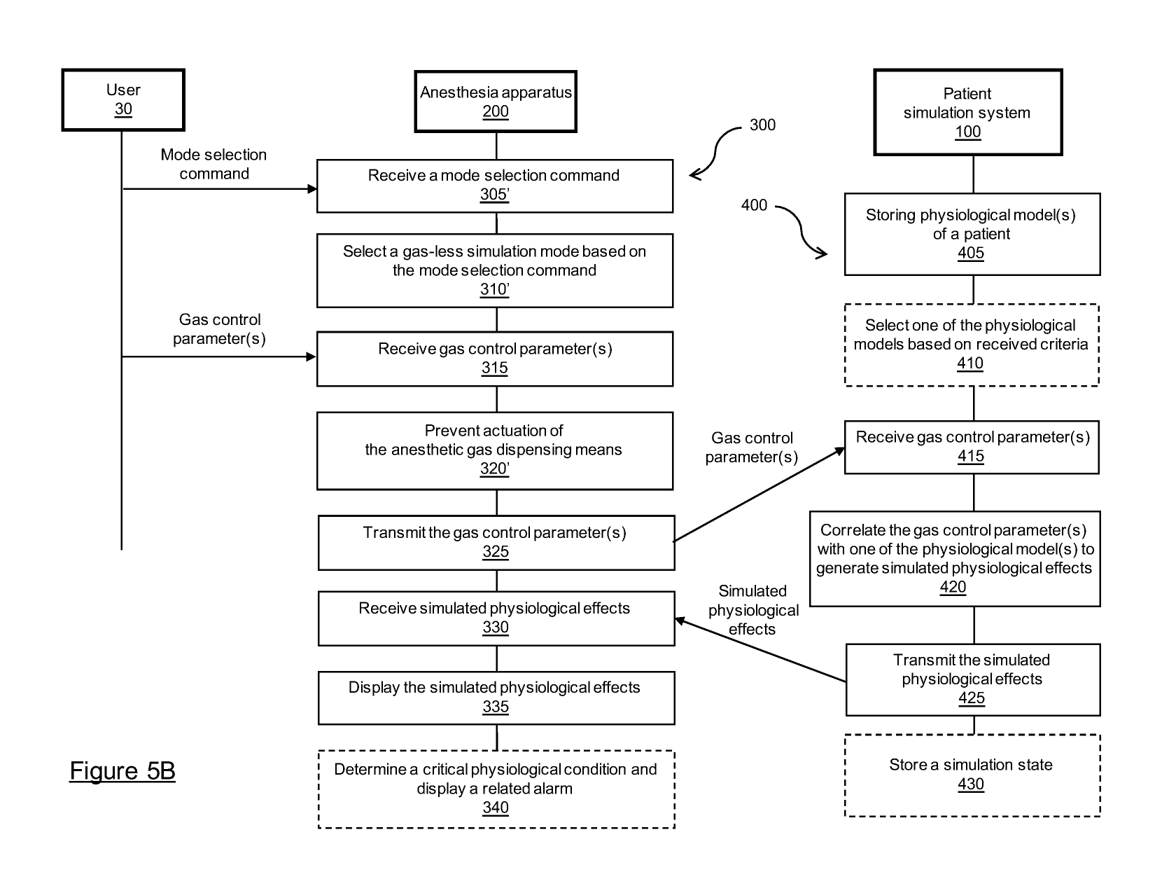

Reference is now made concurrently to FIGS. 1, 2, 3, 5A and 5B. A patient simulation system 100 and an anesthesia apparatus 200 are represented in FIGS. 1, 2 and 3. A method 400 for operating the patient simulation system 100 is represented in FIG. 5B. A method 300 for operating the anesthesia apparatus 200 is represented in FIGS. 5A and 5B.

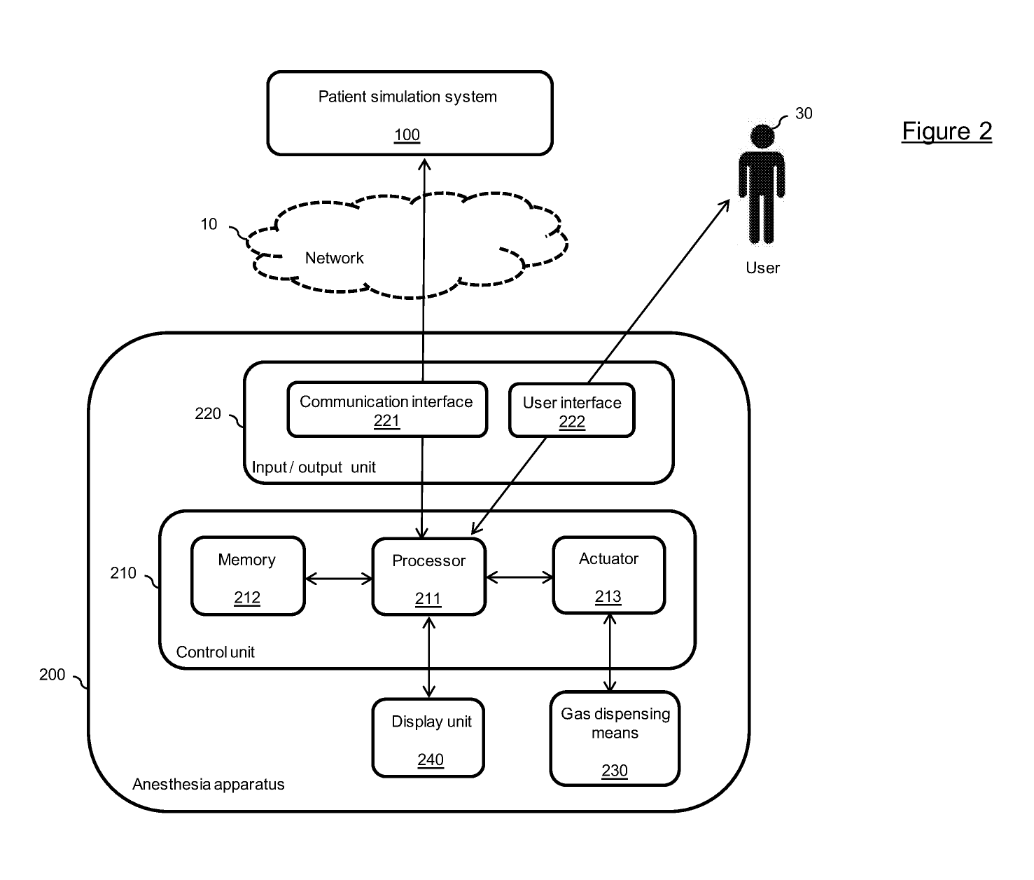

Referring more particularly to FIGS. 1 and 2, details of the anesthesia apparatus 200 are represented. The anesthesia apparatus 200 is a standard anesthesia apparatus as is well known in the art, which delivers anesthetic gas(es) to a patient when operating in a gas-dispensing mode. However, the anesthesia apparatus 200 is further adapted to interact with the patient simulation system 100 when operating in a gas-less simulation mode.

The anesthesia apparatus 200 comprises a control unit 210. The control unit comprises one or more processors 211. A single processor 211 is represented in FIGS. 1 and 2 for simplification purposes. Each processor 211 may further have one or several cores. Each processor 211 is capable of executing instructions of computer program(s), in particular for controlling actuator(s) 213.

The control unit 210 comprises memory 212 for storing instructions of the computer program(s) executed by the processor(s) 211, data generated by the execution of the computer program(s), data received via an input/output unit 220, etc. The control unit 210 may comprise several types of memories, including volatile memory, non-volatile memory, etc.

The control unit 210 comprises one or more actuators 213 for actuating gas dispensing means 230 of the anesthesia apparatus 200. A single actuator 213 is represented in FIGS. 1 and 2 for simplification purposes. Examples of such actuator(s) 213 include mechanical actuators, pneumatic actuators, hydraulic actuators, electric actuators, etc.

The anesthesia apparatus 200 comprises an input/output unit 220. The input/output unit 220 comprises a communication interface 221 for exchanging data with other devices through communication links, generally referred to as the network 10. In particular, the anesthesia apparatus 200 exchanges data with the patient simulation system through the communication interface 221. The communication interface 221 comprises at least one of the following: an Ethernet communication interface, a wireless communication interface (e.g. Wi-Fi, Bluetooth or cellular interface), and a combination thereof. Furthermore, information exchange via the communication interface 221 may support known standards like Health Level Seven (HL7), etc.; or more recent ones currently still in development like Open-Source Integrated Clinical Environment (Open ICE), Open System and Device Connectivity (OpenSDC), etc.

The input/output unit 220 also comprises at least one user interface 222 for allowing a user 30 (a healthcare professional, technician, trainee, instructor, tester, person responsible for defining a simulation scenario, etc.) to interact therewith, for instance for controlling the gas dispensing means 230 via the actuator(s) 213 of the control unit 210. A single user interface 222 is represented in FIGS. 1 and 2 for simplification purposes. Examples of user interface(s) 222 include a keyboard, a mouse, a trackpad, a touch screen, etc.

The anesthesia apparatus 200 comprises the gas dispensing means 230, which include any component(s) (e.g. mechanical, electric, electronic, pneumatic, hydraulic etc.) providing the capability to dispense one or more anesthetic gas to a patient 20. Such gas dispensing means 230, and their interactions with the actuators(s) 213 under the control of the processor(s) 211 is well known in the art of anesthesia apparatus.

The anesthesia apparatus 200 comprises at least one display unit 240 for displaying data generated by the processor(s) 211, information received via the input/output unit 220, etc. A single display unit 240 is represented in FIGS. 1 and 2 for simplification purposes. Examples of display units 240 include a regular computer screen, a tactile screen, etc.

Instructions of a specific computer program implement the steps of the method 300 when executed by the processor(s) 211 of the anesthesia apparatus 200. The instructions are comprised in a non-transitory computer program product (e.g. memory 212). The instructions provide for operating the anesthesia apparatus 200 in one of a gas-dispensing mode and a gas-less simulation mode, when executed by the processor(s) 211. The instructions of the non-transitory computer program product are deliverable via an electronically-readable media, such as a storage media (e.g. a USB key or a CD-ROM) or the network 10 (through the communication interface 221).

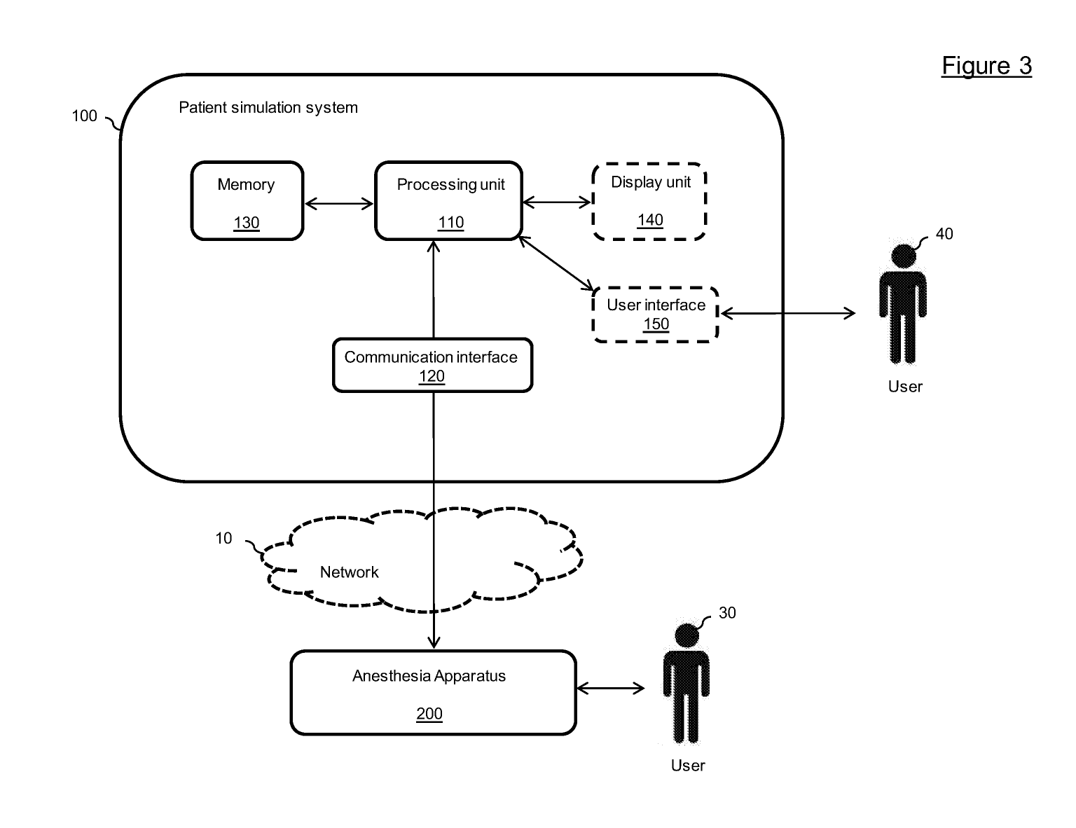

Referring more particularly to FIG. 3, details of the patient simulation system 100 are represented. The patient simulation system 100 is a real time full body patient simulator (e.g. physical mannequin simulating a patient through dedicated hardware and software), or a virtual patient simulator (entirely instantiated with patient simulation software executed by standard or dedicated hardware), capable of executing a real time simulation of a patient. The patient simulation system 100 receives data from the anesthesia apparatus 200, processes the received data to generate simulation data, which are transmitted to the anesthesia apparatus 200. The term gas-less refers to the fact that the patient simulation system 100 interacts with the anesthesia apparatus 200 when the anesthesia apparatus 200 is operating in the gas-less simulation mode, where no anesthetic gas is delivered by the anesthesia apparatus 200.

In the rest of the description, we will refer to the interactions between the patient simulation system 100 and the anesthesia apparatus 200. However, the patient simulation system 100 is not limited to the interactions with the anesthesia apparatus 200, but is also capable of interacting with any type of medical apparatus capable of transmitting gas control parameter(s) and receiving/processing simulated physiological effects generated and transmitted by the patient simulation system 100. The gas control parameter(s) and the simulated physiological effects will be detailed later in the description.

The patient simulation system 100 comprises a processing unit 110, having one or more processors (not represented in FIG. 3 for simplification purposes) capable of executing instructions of computer program(s) for executing the patient simulation. Each processor may further have one or several cores.

The patient simulation system 100 comprises memory 130 for storing instructions of the computer program(s) executed by the processing unit 110, data generated by the execution of the computer program(s), data received via a communication interface 120, etc. The patient simulation system 100 may comprise several types of memories, including volatile memory, non-volatile memory, etc.

The patient simulation system 100 comprises the communication interface 120 for exchanging data with other devices through communication links, generally referred to as the network 10. In particular, the patient simulation system exchanges data with the anesthesia apparatus 200 through the communication interface 120. The communication interface 120 comprises at least one of the following: an Ethernet communication interface, a wireless communication interface (e.g. Wi-Fi. Bluetooth or cellular interface), and a combination thereof. As mentioned previously, information exchange via the communication interface 120 may support standards like HL7, Open ICE, OpenSDC, etc.

The patient simulation system 100 may comprise at least one display unit 140 for displaying data generated by the processing unit 110, information received via the communication interface 120 or a user interface 150, etc. A single display unit 140 is represented in FIG. 3 for simplification purposes. Examples of display units 140 include a regular computer screen, a tactile screen, etc.

The patient simulation system 100 may comprise at least one user interface 150 for allowing a user to interact therewith, for instance for controlling the execution of the patient simulation. A single user interface 150 is represented in FIG. 3 for simplification purposes. Examples of user interface(s) 450 include a keyboard, a mouse, a trackpad, a touch screen, etc.

The patient simulation system 100 is implemented by a dedicated computer or server, or alternatively by a standard desktop computer, laptop or tablet, depending for instance on the computing power required for the processing unit 110 and the capacity required for the memory 130 for executing the patient simulation. In a particular implementation, the patient simulation system 100 operates as a cloud based simulation server, and is remotely controlled by a user device (not represented in the Figures) via the communication interface 120. In this particular implementation, the patient simulation server 100 may not need the display unit 150 and the user interface 150.

Instructions of a specific computer program implement the steps of the method 400 when executed by the processing unit 110 of the patient simulation system 100. The instructions are comprised in a non-transitory computer program product (e.g. memory 130). The instructions provide for operating the patient simulation system 100 adapted to interact with the anesthesia apparatus 200, when executed by the processing unit 110. The instructions of the non-transitory computer program product are deliverable via an electronically-readable media, such as a storage media (e.g. a USB key or a CD-ROM) or the network 10 (through the communication interface 120).

Referring more particularly to FIGS. 5A and 5B, the steps of the methods 300 and 400 are represented. FIG. 5A illustrates the steps of the method 300 when the anesthesia apparatus 200 operates in the gas-dispensing mode, and does not interact with the patient simulation system 100. FIG. 5B illustrates the steps of the methods 300 and 400 when the anesthesia apparatus 200 operates in the gas-less simulation mode, and interacts with the patient simulation system 100. The gas-dispensing mode (illustrated in FIG. 5A) will be described first.

The method 300 comprises the step 305 of receiving a mode selection command from the input/output unit 220. The user 30 uses the user interface 222 of the input/output unit 220 to enter the mode selection command. For example, a graphical user interface is displayed on the display unit 240 by the processor 211 of the control unit 210, allowing the user 30 to select between the gas-dispensing mode and the gas-less simulation mode via the user interface 222. FIG. 5A illustrates the case where the user 30 has selected the gas-dispensing mode. Alternatively, the mode selection command is received via the communication interface 221 of the input/output unit 220, and corresponds to a user interaction of the user 30 with a remote control device (not represented in the Figures). In this particular implementation, the anesthesia apparatus 200 is remotely controlled by a remote control device (e.g. computer, tablet, etc.) operated by the user 30, via commands generated by the remote control device and transmitted to the anesthesia apparatus 200 over the network 10.

The method 300 comprises the step 310 of selecting by the processor 211 of the control unit 210 one of a gas-dispensing mode and a gas-less simulation mode, based on the mode selection command received via the input/output unit 220. FIG. 5A illustrates the case where the gas-dispensing mode is selected.

The method 300 comprises the step 315 of receiving at least one gas control parameter from the input/output unit 220. As mentioned previously with respect to step 305, the gas control parameter(s) is (are) either entered by the user 30 via the user interface 222 or is received via the communication interface 221 (the anesthesia apparatus 200 is remotely controlled by a remote control device operated by the user 30).

Examples of gas control parameters include at least one of the following: type of gas (e.g. oxygen, anesthetic agent), pressure of the gas, flow rate of the gas, composition of the gas for a multi-component gas (e.g. mixture or air with oxygen and anesthetic agent), proportion of each component for a multi-component gas, ventilation mode, ventilator frequency, fresh gas flow, etc.

The method 300 comprises the step 320 of actuating by the control unit 210 the gas dispensing means 230 based on the received gas control parameter(s). More specifically, the processor 211 processes the received gas control parameter(s) and transmits command(s) (e.g. electric command, electronic command, etc.) to the actuator(s) 213. Upon reception of the command(s), the actuator(s) 213 actuate the gas dispensing means 230 for dispensing anesthetic gas(es) to the patient 20, in accordance with the received gas control parameter(s). The interactions between the processor 211, the actuator(s) 213 and the gas dispensing means 230 are well known in the art.

Examples of anesthetic gas which are dispensed to the patient include at least one of the following: Isoflurane, Sevoflurane, Halothane, Enflurane, Desflurane, Nitrous Oxide, etc. These can be administered alone or in any combination thereof up and to include standard respiratory gases like oxygen, nitrogen or fresh gas.

As is well known in the art, the anesthesia apparatus 200 comprises, or is in contact with, sensors (not represented in the Figures for simplification purposes) capable of measuring physiological effects of the anesthetic gas(es) dispensed to the patient 20. The processor 211 receives the measured physiological effects from the sensors, processes the measured physiological effects, and displays information related to the physiological effects on the display unit 240. When operating in the gas-less simulation mode, the role of the patient simulation system 100 is to generate and transmit simulated physiological effects corresponding to the physiological effects measured by the sensors in the gas-dispensing mode.

The gas-less simulation mode (illustrated in FIG. 5B) will now be described. The steps of the methods 300 and 400 are described concurrently, since this mode involves interactions between the anesthesia apparatus 200 and the patient simulation system 100.

In the gas-less simulation mode, steps 305′, 310′, 315 and 320′ of the method 300 correspond to steps 305, 310, 315 and 320 when performed in the gas-dispensing mode illustrated in FIG. 5A. Steps 305′ and 310′ are similar to steps 305 and 310 with the difference that the gas-less simulation mode is selected instead of the gas-dispensing mode.

However, step 320′ is different from step 320. Step 320′ consists in preventing by the control unit 210 actuation of the gas dispensing means 230. For example, when the anesthesia apparatus 200 is in the gas-dispensing mode, the anesthesia apparatus 200 is sometimes positioned in a secure state (e.g. via a command received from the user interface 222, via positioning of a lever of the anesthesia apparatus 200 in a particular position, etc.) where actuation of the gas dispensing means 230 is prevented. The secure state is used for preventing unwanted dispensing of anesthetic gas(es), for example as long as the gas dispensing means 230 are not connected to the patient 20 in a secure manner. Thus, in step 320′, the anesthesia apparatus 200 is positioned in the same secure state as for the gas-dispensing mode. For instance, positioning the anesthesia apparatus 200 in the secure state comprises transmitting by the processor 211 command(s) to the actuator(s) 213, which block the actuation of the gas dispensing means 230.

The method 300 comprises the step 325 of transmitting by the processor 211 the gas control parameter(s) (received at step 315) to the patient simulation system 100 via the communication interface 221.

Before transmission, the gas control parameter(s) may be processed by the processor 211. Examples of such processing include conversion of the gas control parameters to a format adapted to the patient simulation system 100, filtering of the gas control parameters(s), inclusion of additional parameters or settings specific to the anesthesia apparatus 200, etc.

The method 300 comprises the step 330 of receiving simulated physiological effects from the patient simulation system 100 via the communication interface 221, in response to the transmission at step 325 of the gas control parameter(s). The received simulated physiological effects correspond to those transmitted at step 425 of the method 400.

Examples of simulated physiological effects include one of the following: heart rate, blood pressure, tidal volume, respiratory frequency, alveolar gas concentrations of respiratory gases and anesthetic agents, concentration of a physiological component of the blood, etc.

The method 300 comprises the step 335 of displaying by the processor 211 the simulated physiological effects (received at step 330) on the display unit 240.

Before display, the simulated physiological effects may be processed by the processor 211. Examples of such processing include conversion of the simulated physiological effects to a format adapted for display on the display unit 240, filtering of the simulated physiological effects, inclusion of additional parameters specific to the anesthesia apparatus 200, etc.

In another example of processing illustrated at step 340 of the method 300, the processor 211 determines a critical physiological condition based on the processing of the simulated physiological effects, and displays an alarm in relation to the critical physiological condition on the display unit 240. Examples of such critical conditions include heartbeat over or below a particular threshold, blood pressure over or bellow a particular threshold, concentration of a physiological component of the blood over or bellow a particular threshold, etc.

The method 300 may include the additional step(s) (not represented in the Figures) of transmitting by the processor 211 configuration data to the patient simulation system 100 via the communication interface 221 and/or receiving by the processor 211 configuration data from the patient simulation system 100 via the communication interface 221. Examples of such configuration data will be detailed later, in relation to the description of the method 400.

The method 400 comprises the step 405 of storing by the processing unit 110 of the patient simulation system 100 at least one physiological model of a patient in the memory 130. A user 40 (a healthcare professional, technician, trainee, instructor, tester, person responsible for defining a simulation scenario, etc.) uses the user interface 150 to create the physiological model(s) of a patient. For example, a graphical user interface is displayed on the display unit 140 by the processing unit 110, allowing the user 40 to create the physiological model(s) of a patient via the user interface 150. Alternatively, the physiological model(s) of a patient is received via the communication interface 120, and corresponds to a user interaction of the user 40 with a remote control device (not represented in the Figures). In this particular implementation, the patient simulation system 100 is remotely controlled by a remote control device (e.g. computer, tablet, smartphone, etc.) operated by the user 40, via commands generated by the remote control device and transmitted to the patient simulation system 100 over the network 10.

An example of a physiological model of a patient comprises the modeling of a plurality of organs, systems and regulatory controls of the body of a patient (e.g. heart, lung, baroreflex, kidney, liver, stomach, organ of toxicity, tumor, etc.), the interactions between these organs and systems, and their response to a set of therapeutic interventions (like the administration of a drug, cardiopulmonary resuscitation massage, external positive pressure ventilation, etc). The physiological model includes data structures describing the plurality of organs and systems, their expected interactions. The physiological model also includes instructions of simulation software(s) which, when executed by the processing unit 110, simulate the expected interactions.

In the case where a plurality of physiological models of a patient are stored in the memory 130, the method 400 comprises the step 410 of selecting one among the plurality of stored physiological models of a patient, based on criteria received via one of the communication interface 120 and the user interface 150. Step 420 of the method 400 is performed with the selected physiological model of a patient. For example, a graphical user interface is displayed on the display unit 140 by the processing unit 110, allowing the user 40 to select one among the plurality of physiological models of a patient the via the user interface 222. Alternatively, the selection of one among the plurality of physiological models of a patient is received via the communication interface 120, and corresponds to a user interaction of the user 40 with a remote control device (not represented in the Figures). The criteria for selecting one among the plurality of stored physiological models of a patient may also be transmitted by the anesthesia apparatus 200. In this last case, the method 200 comprises the additional steps (not represented in the Figures) of determining the criteria (e.g. through an interaction of the user 30 with the user interface 222 of the anesthesia apparatus 200), and transmitting the determined criteria to the patient simulation system 100.

The method 400 comprises the step 415 of receiving at least one gas control parameter from the anesthesia apparatus 200 via the communication interface 120. The received gas control parameter(s) correspond(s) to those transmitted at step 325 of the method 300.

The method 400 comprises the step 420 of correlating by the processing unit 110 the gas control parameter(s) (received at step 415) with one of the physiological model(s) of a patient (stored at step 405) to generate simulated physiological effects.

Instructions of simulation software(s), when executed by the processing unit 110, analyze the received gas control parameter(s) and the stored physiological model to determine the correlations therebetween, and further generate the simulated physiological effects based on the determined correlations. For example, a received flow rate and pressure of oxygen is correlated to a model defining a variation of the heartbeat based on parameters including at least the flow rate and the pressure of oxygen, to generate a corresponding value of the heartbeat. Similarly, the received flow rate and pressure of oxygen is correlated to a model defining a variation of the blood pressure in a particular arteria based on parameters including at least the flow rate and the pressure of oxygen, to generate a corresponding value of the blood pressure in the particular arteria.

The method 400 comprises the step 425 of transmitting by the processing unit 110 the simulated physiological effects to the anesthesia apparatus 200 via the communication interface 120.

The method 400 also comprises the optional step 430 of storing a simulation state in the memory 130. Although represented after step 425 in FIG. 5B, step 430 may also occur after step 415, after step 420, etc. The simulation state is representative of a status of each of the entities of the physiological model of a patient (e.g. status of the organs and systems of the physiological model, status of the interactions between the organs and systems, etc.), at a particular stage of the patient simulation. The simulation step is automatically generated and stored by the processing unit 110. Alternatively, the simulation step is generated and stored by the processing unit 110 following a command received from the user 40 via the user interface 150 (or following a remote command received via the communication interface 120). A plurality of simulation states representative of a complete simulation session, or representative of only a part of a simulation session, are stored in the memory 130. The stored simulation states can be used for performing one of the following tasks: replay of a simulation session for performing a trainee evaluation/debriefing, learning class to one or more trainees, an analysis, debugging of the patient simulation system 100, etc. The stored simulation states can also be used for starting a simulation session at a particular stage, for instance for allowing a trainee to practice this particular stage repeatedly until being able to perform this particular stage satisfyingly. One or more particular simulation states among the plurality of simulation states stored in the memory can be selected by the user 40 via the user interface 150 (or via a remote selection command received via the communication interface 120) for performing one of the previously mentioned tasks.

After performing step 415, the method 400 may comprise the additional step (not represented in the Figures) of further displaying by the processing unit 110 the received gas control parameter(s) on the display unit 140.

Similarly, after performing step 420, the method 400 may comprise the additional step (not represented in the Figures) of further displaying by the processing unit 110 the generated simulated physiological effects on the display unit 140.

The method 400 may include the additional step(s) (not represented in the Figures) of transmitting by the processing unit 110 configuration data to the anesthesia apparatus 200 via the communication interface 120 and/or receiving by the processing unit 110 configuration data from the anesthesia apparatus 200 via the communication interface 120.

The exchange of configuration data between the anesthesia apparatus 200 and the patient simulation system 100 can be implemented in the form of a handshake initiated by one of the anesthesia apparatus 200 and the patient simulation system 100. For example, the anesthesia apparatus 200 transmits configuration data to the patient simulation system 100, and receives in response configuration data from the patient simulation system 100. The configuration data transmitted by the patient simulation system 100 may depend on the configuration data received from the anesthesia apparatus 200. Alternatively, the anesthesia apparatus 200 transmits a request for configuration data to the patient simulation system 100, and receives in response configuration data from the patient simulation system 100. Then, the anesthesia apparatus 200 transmits configuration data to the patient simulation system 100 which depend on the configuration data received from the patient simulation system 100. The roles of the anesthesia apparatus 200 and the patient simulation system 100 in the handshake may be inverted. The request for configuration data may be transmitted in a broadcast mode, for example to allow the anesthesia apparatus 200 to discover a patient simulation system 100 that is not known in advance Alternatively or complementarily, the configuration data are transmitted in a broadcast mode, for example to allow the patient simulation system 100 to advertise its capabilities to a plurality of anesthesia apparatus 200.

Examples of configuration parameters transmitted by the anesthesia apparatus 200 to the patient simulation system 100 include: a unique identifier of the anesthesia apparatus 200, model of the anesthesia apparatus 200, communication protocol(s) supported by the anesthesia apparatus 200 for exchanging data with the patient simulation system 100, IP address and/or communication ports used by the anesthesia apparatus 200 for exchanging data with the patient simulation system 100, list of gas control parameters generated by the anesthesia apparatus 200, list of simulated physiological effects supported by the anesthesia apparatus 200, etc.

Examples of configuration parameters transmitted by the patient simulation system 100 to the anesthesia apparatus 200 include: a unique identifier of the patient simulation system 100, communication protocol(s) supported by the patient simulation system 100 for exchanging data with the anesthesia apparatus 200, IP address and/or communication ports used by the patient simulation system 100 for exchanging data with the anesthesia apparatus 200, list of gas control parameters supported by the patient simulation system 100, list of simulated physiological effects generated by the patient simulation system 100, etc.

The operations of the patient simulation system 100 and the anesthesia apparatus 200 (when operating in the gas-less simulation mode) are adapted based on the configuration data exchanged between the two equipment. In particular, the generation of the simulated physiological effects by the patient simulation system 100 (step 420 of the method 400) can be adapted to a particular type of anesthesia apparatus 200, based on at least some of the configuration data received from the particular type of anesthesia apparatus 200.

The criteria for selecting one among the physiological models used at step 410 of the method 400 may be included in the configuration data transmitted by the anesthesia apparatus 200 to the patient simulation system 100.

The exchange of configuration data may consist in a multistep negotiation protocol, to adapt the simulated physiological effects generated by the patient simulation system 100 and the gas control parameters generated by the anesthesia apparatus 200 to one another.

The exchange of configuration data may occur at startup of the anesthesia apparatus 200 and the patient simulation system 100, each time the gas-less simulation mode is selected at the anesthesia apparatus 200, upon a particular trigger (e.g. a particular user interaction) occurring at the anesthesia apparatus 200 and/or at the patient simulation system 100, etc.

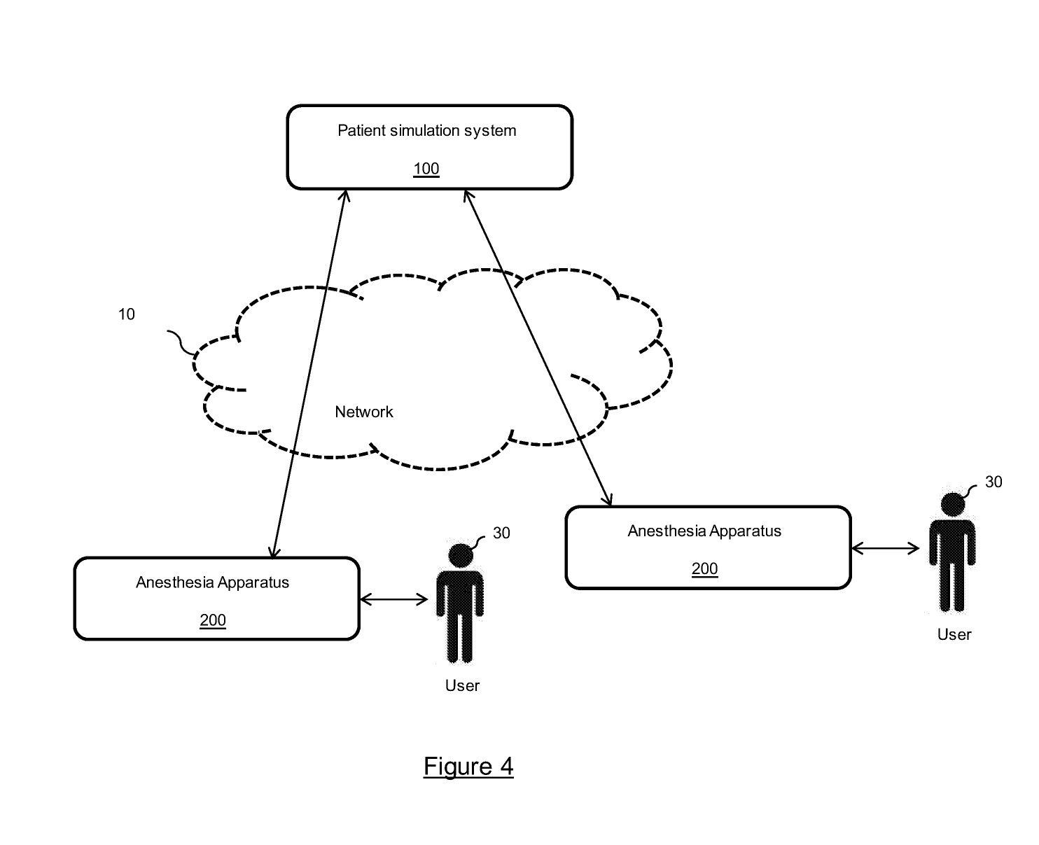

Reference is now made concurrently to FIGS. 1, 2, 3, 4 and 5B. FIG. 4 illustrates a patient simulation system 100 capable of interacting with a plurality of anesthesia apparatus 200. Although only two anesthesia apparatus 200 are represented in FIG. 4, the number of anesthesia apparatus 200 supported in parallel by the patient simulation system 100 depends only on its capabilities (e.g. processing power of the processing unit 110, memory capacity of the memory 130, throughput of the communication interface 120, etc.).

The processing unit 110 of the patient simulation system 100 receives respective gas control parameter(s) from the plurality of anesthesia apparatus 200 (as per step 415 of the method 400). For each particular anesthesia apparatus 200 among the plurality of anesthesia apparatus, the processing unit 110 correlates the gas control parameter(s) received from the particular anesthesia apparatus 200 with one of the at least one physiological model of a patient, to generate simulated physiological effects (as per step 420 of the method 400). The generated simulated physiological effects are transmitted by the processing unit 110 to the particular anesthesia apparatus 200 (as per step 425 of the method 400).

Although the present disclosure has been described hereinabove by way of non-restrictive, illustrative embodiments thereof, these embodiments may be modified at will within the scope of the appended claims without departing from the spirit and nature of the present disclosure.

What is claimed is:

1. An anesthesia apparatus adapted for operating in one of a gas-dispensing mode and a gas-less simulation mode, the apparatus comprising:
gas dispensing means;
an input/output unit comprising a communication interface for:
receiving a mode selection command; and
receiving at least one gas control parameter;
a control unit for:
selecting one of a gas-dispensing mode and a gas-less simulation mode based on the received mode selection command;
when the gas-dispensing mode is selected, actuating the gas dispensing means based on the received at least one gas control parameter; and
when the gas-less simulation mode is selected:
preventing actuation of the gas dispensing means; and
transmitting the at least one gas control parameter to a patient simulation system via the communication interface.

2. The anesthesia apparatus of claim 1, wherein the control unit processes the at least one gas control parameter before transmission to the patient simulation system, the processing comprising at least one of the following: conversion of the at least one gas control parameter to a format adapted to the patient simulation system, filtering of the at least one gas control parameter, and inclusion of additional parameters specific to the anesthesia apparatus.

3. The anesthesia apparatus of claim 1, when the control unit further:
receives simulated physiological effects from the patient simulation system via the communication interface; and
displays the simulated physiological effects on a display unit of the anesthesia apparatus.

4. The anesthesia apparatus of claim 3, wherein the control unit processes the simulated physiological effects before display on the display unit, the processing comprising at least one of the following: conversion of the simulated physiological effects to a format adapted for display on the display unit, filtering of the simulated physiological effects, and inclusion of additional parameters specific to the anesthesia apparatus.

5. The anesthesia apparatus of claim 4, wherein the control unit determines a critical physiological condition based on the processing of the simulated physiological effects and displays an alarm in relation to the critical physiological condition on the display unit.

6. The anesthesia apparatus of claim 1, wherein the communication interface comprises at least one of the following: an Ethernet communication interface, and a wireless communication interface.

7. The anesthesia apparatus of claim 1, wherein the input/output unit comprises a user interface for receiving at least one of the mode selection command, and the at least one gas control parameter.

8. The anesthesia apparatus of claim 1, wherein at least one of the mode selection command and the at least one gas control parameter are received via the communication interface from a remote control device.

9. The anesthesia apparatus of claim 1, wherein the at least one gas control parameter comprises at least one of the following: type of gas, pressure of the gas, flow rate of the gas, composition of the gas for a multi-component gas, proportion of each component for a multi-component gas, ventilation mode, ventilator frequency, and fresh gas flow.

10. The anesthesia apparatus of claim 1, wherein the simulated physiological effects comprise at least one of the following: heart rate, blood pressure, tidal volume, respiratory frequency, alveolar gas concentrations of respiratory gases and anesthetic agents, concentration of a physiological component of the blood.

11. The anesthesia apparatus of claim 1, wherein the control unit further transmits configuration data to the patient simulation system via the communication interface.

12. The anesthesia apparatus of claim 11, wherein the configuration data comprise at least one of the following: a unique identifier of the anesthesia apparatus, a model of the anesthesia apparatus, one or more communication protocols supported by the anesthesia apparatus for exchanging data with the patient simulation system, a list of gas control parameters generated by the anesthesia apparatus, a list of simulated physiological effects supported by the anesthesia apparatus.

13. The anesthesia apparatus of claim 1, wherein the control unit further receives configuration data from the patient simulation system via the communication interface.

14. The anesthesia apparatus of claim 13, wherein the configuration data comprise at least one of the following: a unique identifier of the patient simulation system, one or more communication protocols supported by the patient simulation system for exchanging data with the anesthesia apparatus, a list of gas control parameters supported by the patient simulation system, a list of simulated physiological effects generated by the patient simulation system.

15. A method for operating an anesthesia apparatus in one of a gas-dispensing mode and a gas-less simulation mode, the method comprising:
receiving a mode selection command from an input/output unit of the anesthesia apparatus;
selecting by a control unit of the anesthesia apparatus one of a gas-dispensing mode and a gas-less simulation mode based on the received mode selection command;
receiving at least one gas control parameter from the input/output unit;
when the gas-dispensing mode is selected, actuating by the control unit gas dispensing means of the anesthesia apparatus based on the received at least one gas control parameter; and
when the gas-less simulation mode is selected:
preventing by the control unit actuation of the gas dispensing means; and
transmitting by the control unit the at least one gas control parameter to a patient simulation system via a communication interface of the input/output unit.

16. The method of claim 15, further comprising:
receiving simulated physiological effects from the patient simulation system via the communication interface of the input/output unit; and
displaying by the control unit the simulated physiological effects on a display unit of the anesthesia apparatus.

17. The method of claim 16, further comprising:
processing by the control unit the simulated physiological effects;
determining by the control unit a critical physiological condition based on the processing of the simulated physiological effects; and
displaying by the control unit an alarm in relation to the critical physiological condition on the display unit.

18. A non-transitory computer program product comprising instructions deliverable via an electronically-readable media, such as storage media and communication links, the instructions when executed by a processor of a control unit of an anesthesia apparatus provide for operating the anesthesia apparatus in one of a gas-dispensing mode and a gas-less simulation mode by:
receiving a mode selection command from an input/output unit of the anesthesia apparatus;
selecting one of a gas-dispensing mode and a gas-less simulation mode based on the received mode selection command;
receiving at least one gas control parameter from the input/output unit;
when the gas-dispensing mode is selected, actuating by the control unit gas dispensing means of the anesthesia apparatus based on the received at least one gas control parameter; and
when the gas-less simulation mode is selected:
preventing by the control unit actuation of the gas dispensing means; and
transmitting the at least one gas control parameter to a patient simulation system via a communication interface of the input/output unit.

19. The computer program product of claim 18, wherein the instructions when executed by the processor of the control unit further effect:
receiving simulated physiological effects from the patient simulation system via the communication interface of the input/output unit; and
displaying the simulated physiological effects on a display unit of the anesthesia apparatus.

20. The computer program product of claim 19, wherein the instructions when executed by the processor of the control unit further effect:
processing the simulated physiological effects;
determining a critical physiological condition based on the processing of the simulated physiological effects; and
displaying an alarm in relation to the critical physiological condition on the display unit.
